(19) United States
(12) Patent Application Publication
(10) Pub. No.: US 2007/0032827 A1
(43) Pub. Date: Feb. 8, 2007

(54) METHOD AND APPARATUS FOR PRODUCING THERAPEUTIC AND DIAGNOSTIC STIMULATION

(76) Inventor: Jefferson J. Katims, Towson, MD (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/463,038

(22) Filed: Aug. 8, 2006

Related U.S. Application Data
(60) Provisional application No. 60/706,038, filed on Aug. 8, 2005.

(51) Int. Cl. A61N 1/00

(52) U.S. Cl. 607/2; 607/40; 607/46

(57) ABSTRACT

Advances have been made in electrical stimulation for medical, diagnostic and therapeutic uses, including cutaneous uses and non-cutaneous uses (such as medical implant devices). The novel systems are based on a modulated continuous symmetric wave-form especially at a high-frequency (1 kHz-50,000 kHz), especially devices including a FPGA or ASCI chip. Such electrical systems finally make possible safe, miniaturized medical devices small enough to be hand-held or implantable. A high-frequency symmetric waveform is used to synthesize a low-frequency sine wave.

### RELATED APPLICATION

This application claims benefit of U.S. provisional application Ser. No. 60/706,038 filed Aug. 8, 2005 titled “Method and apparatus for producing therapeutic and diagnostic stimulation” by Jefferson Katims.

### FIELD OF THE INVENTION

This invention relates to a neuro- and other physiologic stimulation through application of electrical wave-forms, especially to digital/analog neuroselective diagnostic or therapeutic electronic devices considerably miniaturized and especially to enhanced capabilities over conventional devices.

### BACKGROUND OF THE INVENTION

Neuroselective stimulation may be administered to the human nervous system using sound, light or electrical stimuli. It is advantageous to use a neuroselective electrical stimulus in the therapeutic excitation of nervous tissue because various sub-populations of nervous tissue subserve different functions (e.g., excitation or inhibition).

Referring to the Patent Literature:

U.S. Pat. No. 4,305,402 issued Dec. 15, 1981 to J. Katims, for “Method for transcutaneous electrical stimulation,” discloses a method and apparatus for monitoring and obtaining actual bio-electrical characteristics of a subject under predetermined conditions of evoked response stimuli, and by interaction with a computer, applying cutaneous electrical stimulation to the subject, using a signal generator to modify current amplitude and frequency in a direction to achieve bio-electrical characteristics in the subject related to the actual bio-electrical characteristics monitored. The signal generator uses a sinusoidal waveform output. Current Perception Threshold (CPT) is determined using a non-invasive, non-aversive electrical stimulus applied at various frequencies. Frequency ranges of 5-10 Hz, 10-70 Hz, and 70-130 Hz are disclosed.

U.S. Pat. No. 4,503,863 issued Mar. 12, 1985 to J. Katims, for “Method and apparatus for transcutaneous electrical stimulation.”

A manual current perception threshold (CPT) device was commercially developed for the patented Katims technology. Using a manual CPT device (see Katims U.S. Pat. No. 4,305,402 or U.S. Pat. No. 4,503,863), a pair of identical CPT electrodes were placed a specified distance from each other on the skin of the subject to be tested by the technician. The electrodes are generally held in place using a piece of tape. Electrolyte containing conductive gel serves as the conducting medium between the skin to be tested and the electrode surface. It was necessary for the technician to hide the controls of the device from the subject's view, so the subject may not see the output settings of the device. The technician then informed the subject that he/she would manually be slowly increasing the intensity of the CPT stimulus and would ask the subject to report when the stimulus was perceived. When the subject reported perceiving the stimulus the technician would turn off the output of the CPT device. Most commonly, subjects will report their initial perception of the stimulus under one of the electrodes or both of the electrodes in contact with the skin site or in the area of the electrodes. As this is not a naturally perceived stimulus, subjects often have to learn what the stimulus is and, consequently, the initial perceptual report is often well above the actual ultimately determined CPT. The technician then decreases the output intensity in randomly selected decrements and repeatedly presents lower intensities of the stimulus until the subject does not perceive the stimulus. The prior art CPT devices had a three position switch which enabled turning the stimulus either on or off or to a rest (off) position. This switch made a mechanical clicking sound when switched. The technician rotating the knob that clicked between these positions, in order to present the stimulus to the subject. The technician informed the subject that “I am now going to present you with two tests, Test A with a rest and Test B, and I would like you to tell me when you may perceive either Test A or B or whether you cannot perceive either test.” The technician then proceeded to move in a random sequence the output select knob of the CPT device between a true setting, rest setting and a false setting. For example the first two tests would be presented in the sequence Test A was the true setting and the next three tests would be presented in the sequence where Test A was the false setting. By presenting suprathreshold (above threshold) and infra-threshold (below threshold) intensities of stimulus, based on the subject's response, the technician was able to narrow down the threshold between two infra and supra threshold intensity settings. The resolution of the CPT measure was determined by the technician depending upon whether the threshold was determined by large current steps or small steps in current intensity. Using this manual means, the technician was able to approximate the CPT as being the average value between these two intensities. This procedure was repeated by the technician at various stimulus frequencies to determine characteristic CPTs. The technician had to manually write down the CPT value that he/she determined from the testing procedure. These CPT values were then manually entered into a computer software program for statistical evaluation purposes.

The following are also mentioned as background:

J. Katims, D. M. Long, L. K. Y. Ng, “Transcutaneous Nerve Stimulation: Frequency and Waveform Specificity in Humans,” Appl. Neurophysiol. 49: 86-91 (1986).

Katims, J. J., Rouvelas, P., Sadler, B., Weseley, S. A. Current Perception Threshold: Reproducibility and Comparison with Nerve Conduction in Evaluation of Carpal Tunnel Syndrome. Transactions of the American Society of Artificial Internal Organs, Volume 35:280-284, 1989.

J. Katims, D. Taylor and S. Weseley, “Sensory Perception in Uremic Patients,” ASAIO Transactions, 1991, 37:M370-M372.

Katims, J. J., Patil, A., Rendell, M., Rouvelas, P., Sadler, B., Weseley, S. A., Bleecker, M. L. Current Perception Threshold Screening for Carpal Tunnel Syndrome. Archives of Environmental Health, Volume 46(4):207-212, 1991.

D. Taylor, J. Wallace and J. Masdeu, “Perception of different frequencies of cranial transcutaneous electrical nerve stimulation in normal and HIV-positive individuals,” Perceptual and Motor Skills, 1992, 74, 259-264.

U.S. Pat. No. 5,143,081 issued Sep. 1, 1992 to Young et al., for “Randomized double pulse stimulus and paired event analysis.”

U.S. Pat. No. 5,806,522 issued Sep. 15, 1998 to Katims, for “Digital Automated Current Perception Threshold (CPT) determination device and method.”

U.S. Pat. No. 5,851,191 issued Dec. 22, 1998 to Gozani (NeuroMetrix, Inc.), for “Apparatus and methods for assessment of neuromuscular function” for a wrist stimulator.

U.S. Pat. No. 6,029,090 issued Feb. 22, 2000 to Herbst, for “Multi-functional electrical stimulation system.”

U.S. Pat. Application No. US 2002/0055688 published May 9, 2002 by J. Katims, titled “Nervous tissue stimulation device and method,” discloses a method of using a precisely controlled, computer programmable stimulus for neuroselective tissue stimulation that does not leave a sufficient voltage or electrical artifact on the tissue being stimulated that would interfere with or prevent a monitoring system from recording the physiological response with regard to physiological conduction of the tissue being studied. A computer controls the waveform, duration and intensity of the stimulus. A symmetric waveform which is sinusoidal is shown in FIG. 10. Waveforms at 5 Hz and 2 kHz are shown in FIG. 10.

U.S. Pat. No. 6,731,986 issued May 4, 2004 to Mann (Advanced Bionics Corp.) for “Magnitude programming for implantable electrical stimulator.”

U.S. Pat. No. 6,830,550 issued Dec. 14, 2004 to Hedgecock, for “Stair step voltage actuated measurement method and apparatus.” Buttons for 200 Hz, 250 Hz and 5 Hz are shown in FIG. 6. FIGS. 7, 9 show non-symmetric wave-forms.

U.S. Pat. Application No. 2005/192567 published Sep. 1, 2005 by J. Katims, is titled “Nervous tissue stimulation device and method.”

Current Perception Thresholds (CPTs) conventionally have been determined using a transcutaneously applied output stimulus intensity, ranging from 0 to 10 milliamperes, generally with the resolution of 1 to 10 μAmps. More recently models have been developed requiring a 20 mAmp output, which is not a significant modification. These currents mentioned are for a voltage range +/−150 V.

Conventional devices come in a case the size of a medium or large suit case weighing 33 pounds in their cases (14-18 pounds not counting the case). The big size of 33-pound conventional electrical stimulation medical devices besides making them difficult with which to work precludes them from being expanded into applications and uses in which a small size would be required.

### SUMMARY OF THE INVENTION

The present inventor has found the 14-18-pound conventional electrical stimulation medical devices to be undesirably large. Additionally, the present inventor has found the conventional suitcase-sized electrical stimulation medical devices to require too much battery space and/or to be too large a component of the apparatus. The inventor created novel approaches to greatly reduce or eliminate battery space. He also found that the conventional technology could not be used to construct hand-held devices or implantable devices. Additionally, the present inventor desired to find a safer diagnostic/therapeutic/physiologic method (i.e. lower energy stimulus with the same physiologic efficacy as higher energy stimulus). He also desired to generate a less adverse stimulus to enhance patient compliance for follow evaluations or changes in therapeutic/physiologic intervention. By “physiologic” both in vivo and in vitro are meant.

The inventor therefore has invented new technology based on high-frequency symmetric waveforms. Such new technology is practically embodied, for example, in novel circuitry and novel digital controls including digital stimulator controls incorporating a micro controller for which purpose an FPGA or ASIC chip preferably is used.

Electromagnetic power for battery charging may be used to eliminate the need for a battery or a battery charger wire connection in devices for tissue stimulation.

The new discoveries and inventions by the inventor have further led to novel miniaturized devices and novel implantable devices using electrical stimulation for medical, diagnostic and therapeutic applications.

It is an object of the present invention to provide a digital automatic quantitative determination and recording of current or current pain perception thresholds that is both diagnostic and therapeutic and may be used to recommend medical treatment. The present invention may also be used to automatically guide the course of the neuro-diagnostic evaluation.

It is a further object of the invention to provide a therapeutic and/or diagnostic electrical stimulus that uses less charge than conventional devices for both internal and external applications for patients, subjects, animals, etc.

A further object of the invention is to generate a high-quality stimulus of high fidelity with low harmonic distortion.

Another object of the invention is to provide a reduced-size device and battery having equal or better efficiency than conventional large devices.

An additional object of the invention is to make possible applications that otherwise could not be performed without smaller size of a device, such as in, e.g., certain clinical situations where space is limited, medical device implantation, etc.

Another object of the invention is to use an FPGA or ASIC chip to generate a single continuous waveform or multiple waveform stimuli for physiological, diagnostic and therapeutic electrical stimulation.

The invention also has an object high frequency digital generation of waveform providing less distortion and/or a higher fidelity stimulus.

Another object of the invention is to introduce wireless control into neurostimulative and other physio-stimulative technology.

A further object of the invention is to provide a physiologic stimulation device which is useable without a battery in the device during use providing physiologic stimulation.

In a preferred embodiment, the invention provides a medical device comprising: a generator system comprising a field programmable gate array (FPGA) chip or an application-specific integrated circuit (ASIC) chip (such as, e.g., a FPGA chip or ASIC chip that is a high-frequency chip in a range of about 1 kHz to 50,000 kHz; etc.); wherein the generator system generates at least one stimulus (such as, e.g., a generated stimulus which is physioselective; a generated stimulus which is tissue selective) which is a continuous symmetric wave form (such as, e.g., a sine-waveform, a bi-phasic square waveform, a triangular waveform, a modulated high-frequency synthesized waveform, etc.); and at least one electrode or electromagnet system via which the at least one generated stimulus may be administered to a patient or an electrosensitive tissue (such as, e.g., a generated stimulus of a form that can be applied to electrosensitive tissue; a generated stimulus of a form that can be applied to a nerve; a generated stimulus that when applied to a patient elicits no cutaneous sensation and only non-cutaneous sensation; etc.), such as, e.g., a medical device that is hand-held or smaller and/or weighs substantially less than 14-18 pounds and/or has dimensions no bigger than 15 cm by 15 cm by 10 cm such as, e.g., 6 cm by 6 cm by 1 cm; a medical device consisting essentially of the high-frequency FPGA chip or the high-frequency ASIC chip, and only such additional components as are necessary to operate a constant current test when the device is electrically connected to a patient or a tissue; a medical device including a power source (such as, e.g., a battery; a power source comprising an inductance coil; etc.); a medical device powered by an external power source not included in the device; a medical device which is biocompatilized and implantable into a human or animal; etc.

In another preferred embodiment, the invention provides a medical device comprising: a generator system that generates a particular harmonic frequency (such as, e.g., a particular harmonic frequency of biological interest; a particular harmonic frequency of physiological interest; a particular harmonic frequency that is physioselective (such as, e.g., a particular harmonic frequency that is neuroselective among a subpopulation of A, B and C nerve fibers); a particular harmonic frequency that is tissue selective; etc.) by maximizing at least two or more different frequencies which differ from the particular harmonic frequency; and at least one electrode or electromagnet system via which the particular harmonic frequency may be administered to a patient or an electrosensitive tissue; such as, e.g., a medical device wherein the particular harmonic frequency is capable of stimulating different tissue types (such as, e.g., stimulating small diameter nerve fibers); etc.

The invention in another preferred embodiment provides a miniaturized medical device for generating a stimulus receivable by electrosensitive tissue, comprising: a stimulus-generating system that generates stimuli (such as, e.g., a symmetric wave-form(s) (such as, e.g., a continuous, symmetric wave-form(s); etc.)); and an electrode or electromagnet system through which the stimulus can be delivered to electrosensitive tissue, wherein the device is a size that is hand-held or smaller; such as, e.g., a miniaturized medical device having a weight substantially less than 14-18 pounds; a medical device without presence of any of: a Johnson counter or Decade counter; a high-speed semi-conductor CMOS flip flop chip, an analog multiplexer chip, a switched capacitor filter microchip and a surface mount 0.1 μFarad electrolytic bypass capacitor; a miniaturized medical device implantable into a human or animal; etc.

The invention in another preferred embodiment provides a method of generating medically-useable electrical stimulation, comprising: within a device of a size that is hand-held or smaller, generating (such as, e.g., a generating step that comprises operating a high-frequency FPGA chip or a high-frequency ASIC chip; etc.) at least one electrical stimulus having a continuous symmetric waveform (such as, e.g., a continuous symmetric waveform having a high frequency in a range of about 1 kHz to 50,000 kHz; etc.); providing the at least one electrical stimulus to an electrode or electromagnet system wherein the electrode or electromagnet system is contactable with an electrosensitive tissue or a patient.

In another preferred embodiment, the invention provides a method of electrically stimulating electrosensitive tissue, comprising: within a device of a size that is hand-held or smaller, generating at least one electrical stimulus having a continuous symmetric wave form (such as, e.g., generating at least one electrical stimulus having a high-frequency in a range of about 1 kHz to 50,000 kHz); and applying the at least one electrical stimulus to an electrosensitive tissue (such as, e.g., an applying step that comprises contacting a stimulating electrode with a patient who may be human or animal or with an electrosensitive tissue; etc.); such as, e.g., methods wherein the step of applying the at least one electrical stimulus is performed cutaneously; methods wherein the step of applying the at least one electrical stimulus is performed non-cutaneously; methods wherein the step of applying the at least one electrical stimulus results in nerve or tissue stimulation; etc.

### BRIEF DESCRIPTION OF THE DRAWINGS

FIG. 1 depicts a sinusoid wave form, such as discussed in Method 1 of Inventive Example 1A.

FIGS. 1A-1C depict exemplary symmetric wave-forms for use in the invention. FIG. 1A is a sinusoid waveform. FIG. 1B is a biphasic square waveform. FIG. 1C is a triangular wave-form.

FIG. 1D is an illustration of an exemplary embodiment of an inventive system in which electrodes are connected to a subject's finger, with the subject operating the device via a hand held personal computer (PC) which communicates to the stimulator through wireless technology.

FIG. 2 is an illustration of an inventive apparatus in an exemplary embodiment.

FIG. 3 is a Field Programable Gate Array (FPGA) or ASIC Chip showing Pin connections which may be used in an exemplary embodiment of the invention;

FIG. 4 is a block diagram illustration of an electrical stimulation system in an inventive embodiment;

FIG. 5 is a schematic diagram of a power supply which may be used in an embodiment of an inventive system;

FIG. 6 is a schematic diagram of a microcontroller section useable in an embodiment of an inventive system;

FIG. 7 illustrates a stimulating electrode placed on the back of a subject's hand in an embodiment of using the invention.

FIG. 8 is a schematic diagram of a Battery Integrator and Clipping detection circuit which may be used in an embodiment of an inventive system;

FIG. 9 is a schematic diagram of a Digital waveform synthesizer which may be used in an embodiment of an inventive system;

FIG. 10 is an illustration of an exemplary back panel of an exemplary inventive device;

FIG. 11 is a schematic diagram of an output stage which may be used in an exemplary embodiment of the invention.

FIG. 12 is a schematic diagram of a battery charger circuit which may be used in an embodiment of an inventive system;

### DETAILED DESCRIPTION OF THE PREFERRED EMBODIMENT OF THE INVENTION

The invention may be appreciated further with reference to the figures, without the invention being limited to the figures.

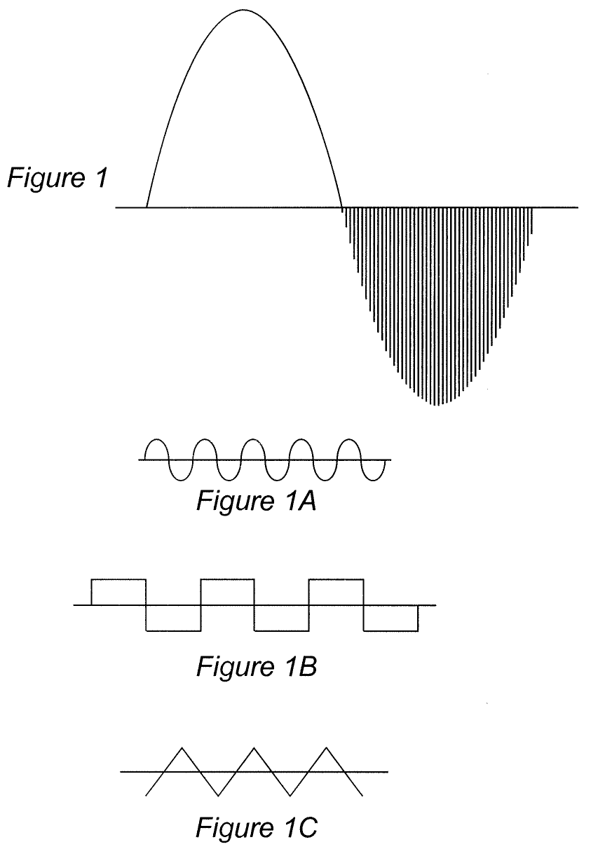

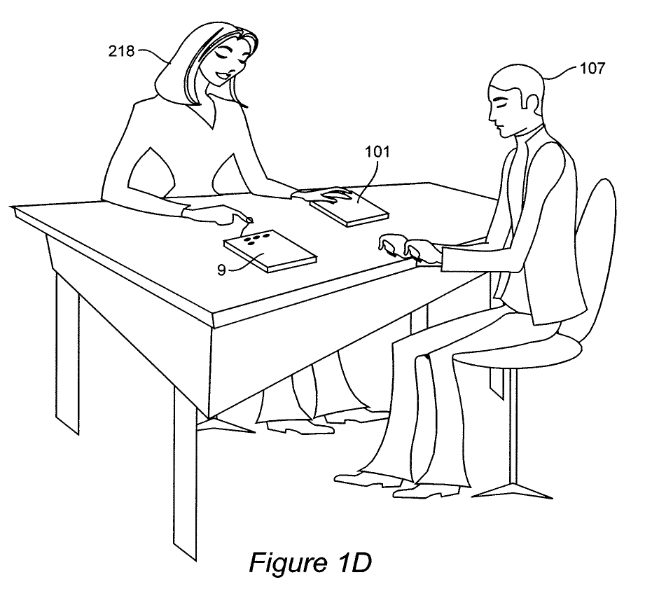

In the inventive devices, apparatuses, methods, products and systems (including but not limited to methods and apparatuses for producing diagnostic and therapeutic electrical stimulation), a symmetric wave-form is used. Examples of a symmetric wave-form is, e.g., a sinusoid wave-form (see FIG. 1A), a biphasic square wave-form (see FIG. 1B), a triangular wave-form (see FIG. 1C), etc. The symmetric wave-forms shown in FIGS. 1A-1C are illustrative and the invention is not limited thereto. “Symmetric” as used herein for a wave-form means a wave-form described by points (x, y) when x is between 0-180 degrees and then when x is between 180-360 degrees, by points (x+180, −y); that is, the wave-form over 0-180 degrees is repeated over 180-360 degrees with the only difference being that y becomes (−y) in the 180-360 degree phase.

The preferred waveform is a continuous symmetric fundamental and related harmonic waveforms, i.e., sinusoid waveforms of specific amplitudes or frequencies (also referred to as harmonics). Preferably the symmetric wave form is continuous, without or with minimal inter-pulse intervals.

The duration need not be continuous in all embodiments of the invention; for example, amplitude modulated high frequency steps may be used to construct a waveform of appropriate frequency for application to tissue, a subject, etc. For example, same-duration steps may be used, of differing amplitude, balanced out over time, to construct a digital version of an analog stimulus.

The time during which at least one wave form is generated is, e.g., a time in range of about 0.1 second to several minutes depending on the application.

In the invention, there may be used either a single waveform or multiple waveforms of electrical stimulation simultaneously to achieve a desired electro-physiological response. An example of using multiple waveforms simultaneously is, e.g., using a 2000 Hz waveform simultaneously with a 2040 Hz waveform. When multiple waveforms are used simultaneously, they should be used so that the resulting sum or difference waveform has the optimal energy at the desired frequency.

Using high frequency stimulation with low frequency modulation, makes possible to achieve the same physiological effect with less electrical charge. This is desirable as it serves to reduce the charge exposure to the subject (human or animal) or tissue being stimulated. Advantageously, high frequency wave forms may be generated and manipulated to generate specific low frequency sinewave harmonics to minimize charge expenditure which allows for safer stimulation as well as prolonging battery life of a battery powering the system.

“Stimulation” herein means excitatory or inhibitory stimulation.

In the invention, electrical stimulation (comprising a symmetric wave-form) is administered to the tissue. With electrical stimulation administered to the human nervous system, there are factors which must be satisfied. When administering electrical stimulation to tissue, it is necessary to keep the charge density of the stimulus to a minimum to avoid causing tissue damage. In addition to comfort and safety concerns, for implantable or small medical device engineering, a low charge electrical stimulus also extends the operational life of its battery power source.

In the invention, the symmetric wave-forms used are high-frequency. The present inventor has found that high-frequency wave forms may be used to generate specific low frequency sine wave harmonics, thereby minimizing charge expenditure and prolonging battery life.

In generating the high-frequency symmetric wave form, preferably the invention is practiced using a high-frequency FPGA chip or a high-frequency ASIC chip. A preferred example of a high-frequency FPGA or ASIC chip to use is a chip having a normal operational range up to about 50 MHz.

A power source for an inventive tissue stimulator device is selected according to the application. Advantageously, in certain embodiments, use of electromagnetic power or battery charging may be used to eliminate the need for a battery or a battery charger wire connection, providing tissue stimulation devices which do not require a battery within the device while the device is being used to provide tissue stimulation. For example, an inductance coil charging station may be used for externally powering an inventive physiologic stimulator device, eliminating the need for wires between a stimulator device and its power source. By using a transducer mechanism, the need for a battery for wiring to a power source may be advantageously eliminated, which is advantageous for powering an implantible device that may be powered by an external power source.

Examples of what may be stimulated according to the invention include, e.g., nerves and tissue. Advantageously, a subpopulation of nerves or a subpopulation of tissue may be selectively stimulated, such as, e.g., stimulating a small diameter nerve fiber, which nerve fiber may be within a living patient or subject, by applying to the nerve fiber at least one continuous symmetric wave form (such as, e.g., a continuous symmetric wave form generated using at least one high frequency in a range of 1 kHz to 50,000 kHz).

When operating stimulator devices embodying the invention, preferably an amount of current required is minimized and small, such as requiring less than 20 mAmp. The amount of current required generally depends on the application. For example, a routine clinical application may require, e.g., 10 mAmp or less and an application in which anaesthesia is used may require 20 mAmp or less.

Regarding type of current used in the invention, current may be direct current (using migration of ions to anode and cathode) or constant current (using an equivalent charge at each electrode), with constant current being preferred. Using constant current is preferred as being physiologically safer and also for advantageously accounting for variations in impedance or resistance of tissue (such as changes that occur when perspiration occurs, when drying occurs, etc.).

Voltage of devices (with or without a battery) embodying the invention may be customized according to the application. For example, in some applications a conventional transformer (a wire wrapped toroid) may be used or the toroid may be built into the device, in which the voltage is a function of the wraps, in commercially available sizes. Alternately, customized toroids may be formed for delivering particular voltages. Alternately, a custom-wrapped electro-conductive coil may be used. A wire wrapped toroid or custom-wrapped electro-conductive coil, when used, may be used in the stimulator device or outside the stimulator device in cooperation with the device.

Preferably the invention is practiced using stimulator devices configured into relatively small sizes. An example of a relatively small sized weight is a weight substantially less than 14-18 pounds (of which a weight of 1 pound or less is a preferred example). An example of relatively small sized dimensions for an inventive stimulator device are dimensions no bigger than 15 cm by 15 cm by 10 cm, preferably 6 cm by 6 cm by 1 cm.

The electrical stimulus provided by the invention may be applied, e.g., to electrosensitive tissue, nerves, etc., and subpopulations thereof. The electrosenstive tissue, nerves, etc. receiving the stimulus may be, e.g., within a human, within an animal, etc. Tissue stimulation according to the invention may be provided by applying an electrode or electromagnet system cutaneously or non-cutaneously to a tissue.

An electrode system includes at least two conductors, such as a stimulator electrode and a dispersion electrode. Examples of placement for applying a stimulator electrode are, e.g., bladder, stomach, shoulder, pancreas, bowel, gall bladder, bile flow, etc. The invention may be used for application of a stimulus to the tissue for, e.g., controlling internal muscular action, bladder therapy, stomach therapy, evoking selective insulin release for treatment of diabetes, other pancreas therapy, etc.

For the mentioned internal placements (such as bladder, stomach and shoulder placements, etc.) using a stimulator device with an external charger is preferred. An example of placing a dispersion electrode is, e.g., a foot of a patient to whom a stimulator electrode has been applied elsewhere in a region to be stimulated. Generally a stimulator electrode is relatively small and a dispersion electrode is relatively large.

Alternately to an electrode system, a system of at least one electromagnet may be used, to provide electromagnetic stimulation. An example of an electromagnet system to use in practicing the invention is a circular ferrous bracelet which may be placed around a wrist, with an electrical connection to an output of an inventive stimulator device, with current generating an electromagnetic field.

The present invention may be used, e.g., in diagnostic uses, therapeutic uses, medical uses, etc.

The invention may be further appreciated with reference to the following Examples, without the invention being limited to the examples.

### COMPARATIVE EXAMPLE 1

The medical electrical stimulators described in Katims U.S. Pat. Nos. 4,305,402; 4,503,863 and 5,806,522 are the size of large lap top computers.

### Comparative Example 1A (Neurotron, Inc.'s Neurometer)

Neurotron, Inc.'s device includes the following components:

(1) Four (4)-4-stage divide-by-8 Johnson counters or Decade Counters/Dividers similar to the medically certified counter microchips manufactured by Fairchild Industries;

(2) Two (2) advanced high-speed semi-conductor CMOS Flip Flop microchips (e.g., STMicroelectronics);

(3) One (1) analog multiplexer integrated circuit microchip, similar to the medically certified multiplexer microchips manufactured by Fairchild Industries;

(4) One (1) fourth (4^{th}) order switched capacitor filter microchip (National Semiconductor); and

(5) Ten (10) surface mount 0.1μ Farad electrolytic bypass capacitors. Capacitors included in the device of this Comparative Example 1 occupy approximately 25% of the circuit board.

Components (1)-(5) require approximately 5.74 cm² of circuit board space.

Comparative Example 1 is powered by a valve-regulated lead acid battery, such as Panasonic LC-R067R2P which has expected trickle life of 3-5 years at 25 degrees C., dimensions of about 151 mm by 134 mm by 94 mm and total height of 100 mm, weighing approximately 1.26 kg.

Neurotron, Inc.'s data published about 2002-3 on its website for its table-top size Neurometer® CPT device report CPT frequencies 5 Hz, 250 Hz and 2000 Hz, for healthy mean CPT values, 1 CPT=10 microAmperes.

This machine has weight of about 14.25 pounds and has dimensions of about 15.5 inches (L), 11.5 inches (W) and 5 inches (H). This machine has a remote box weighing about 1.9 lbs with dimensions about 5 inches (L), 5 inches (W) and 4 inches (H), with a cable connection with the main unit. Other features include: a large and small knob; LCD display; 18 switches with built in LD (9 inches by 1 inch) and LEDs, a printer, and a remote patient response box which includes 4 switches and 4 LEDs; and the following connectors: 1 telephone (TELCO 6-4) electrode; 1 remote box (TELCO 8-8); 1 printer power and battery charger; DC connectors (2.1-2.5 mm); 2 serial ports DB-9.

### INVENTIVE EXAMPLE 1

Human and animal nervous tissue and other electrosensitive tissue (e.g., muscular) is capable of discriminating the harmonic or spectral components of an electrical stimulus and responding selectively to these components of the stimulus. (Katims, U.S. Pat. No. 5,806,522; Katims, U.S. Pat. No. 4,503,863; Katims, U.S. Pat. No. 4,305,402; Katims, J. J., Long, D. M., Ng, L. K. Y., “Transcutaneous Nerve Stimulation (TNS): Frequency and Waveform Specificity in Humans, Applied Neurophysiology, vol. 49: 86-91, 1986; Katims, J. J., “Electrodiagnostic Functional Sensory Evaluation of the Patient with Pain: A Review of the Neuroselective Current Perception Threshold (CPT) and Pain Tolerance Threshold (PTT),” Pain Digest, vol. 8(5), 219-230, 1998; Kog, K., Furue, H., Rashid, M., Takaki, A., Katafuchi, T., Yoshimura, M., “Selective activation of primary afferent fibers evaluated by sine-wave electrical stimulation,” Molecular Pain, vol. 1:13, 2005.)

The sinusoid waveform represents a pure harmonic stimulus. Various frequencies (e.g., 5 Hz, 100 Hz, 2000 Hz) of a sinusoid waveform electrical stimulus selectively excite specific sub-populations of nerve tissue. At frequencies above 5000 Hz usually there is no direct electrically evoked tissue response to the stimulus. If a 5000 Hz stimulus is administered with sufficient intensity, it is possible to burn the skin before any electrophysiological or sensory response is evoked.

Although the present invention refers to a continuous waveform or continuous waveforms of electrical stimulation with durations typically greater than 1 second and as long as several minutes for the purposes of illustrating the functioning of this invention the present paragraph refers to just a single cycle or 360 degrees of a sine waveform stimulus. A 360 degree 5 Hz sine waveform with a peak amplitude of 1 mAmp has 400 times the electrical charge as compared to a 360 degree 2000 Hz sine waveform of the same amplitude. Each sinusoid waveform has a characteristic duration, 0.5 msec and 200 msec for 2000 Hz and 5 Hz waveforms respectively. Thus it is preferable to use the high frequency stimulus because it has a lower electrical charge. However, a 5 Hz sinusoid waveform stimulus is capable of selectively exciting small diameter nerve fibers and this type of stimulation may be therapeutically or diagnostically indicated (e.g. for relief from pain or inhibition of tremor or evaluation of nerve dysfunction) when it is necessary to selectively modulate the functioning of the small diameter nerve fibers. The 2000 Hz sine waveform in contrast has no ability to stimulate the small diameter nerve fibers. (Koga et al. 2005.) It is possible, however, to take advantage of the ability of the nervous system to discriminate harmonics and detect differences in harmonics. For example if a 5 Hz stimulus is required this may be administered using either of the two following methods:

Method 1

The sinusoid waveform is digitally synthesized from consecutive steps or high frequency pulses at varying intensities based in their temporal position in the sine waveform. In FIG. 1, an illustration of 360 degrees of a sinusoid waveform, the first 180 degrees illustrates a pure 5 Hz stimulus and the second 180 degrees illustrates a digitally synthesized sinusoid waveform composed of high frequency pulses or steps. In this example, the amplitude of the pulses or steps equals the sine of the angle or duration of the sinewave. For example, consider the pulses that comprise the first 180° of a 5 Hz sinewave (100 msec) set at a peak intensity of 1.0 mAmp. At 45° or 25 msec the pulse amplitude is 0.5 mAmp. and at 90° or 50 msec the pulse amplitude is 1.0 mAmp., at 135° or 75 msec the amplitude is 0.5 mAmp. and at 180° or 100 msec the amplitude is zero. These high frequency pulses or individual steps could be of such a brief duration as to be incapable of exciting the tissue being tested or treated if presented individually or presented at an unmodulated intensity.

To stimulate approximately 1 square cm of skin on the healthy finger tip of a person requires on average 0.5 mAmp. (peak intensity) of a 5 Hz sinusoid waveform stimulus for approximately 3 seconds (depolarization period or 180°=100 msec) to evoke sensation, whereas a 2000 Hz sinusoid stimulus (depolarization period or 180°=0.25 msec) at the same site has an average threshold of 2.25 mAmp when applied for approximately 1 second. The inventor through his research has determined that using modulated 0.25 msec sinusoid pulses with 0.25 msec rest periods between the pulses (or 180° of 2000 Hz sinewave) to generate a 5 Hz stimulus is similarly effective as a continuous 5 Hz depolarization as shown on the left half of the sinewave in FIG. 1 in the evocation of 5 Hz sensation. The digitally generated stimulus uses less charge than the continuous stimulus.

An important advantage of the present invention is to minimize the current required for diagnostic or therapeutic efficacy. A major advantage of the reduced current requirement of the present invention is to permit significant decreases in the battery and other component size requirements over conventional devices, which permits greater miniaturization and provides longer battery life.

Method 2

A second digital means for the generation of neuroselective or tissue selective stimulation involves a carrier frequency. For example a 2000 Hz stimuli may simultaneously be administered with a 2005 Hz stimulus and the 5 Hz differential harmonic frequency between these two stimuli will be a dominant stimulus.

Methods 1 or 2 of this example may be used to take advantage of neuroselective high frequency digital stimulation to permit the miniaturization of the present invention to be a hand held, inserted or implanted medical device. The present invention provides, e.g., a method and apparatus that uses harmonics of a high frequency electrical stimulus to:
1. Provide neuroselective stimulation.
2. Provide effective low charge stimulus that is safer and potentially less adverse than a high charge stimulus.
3. Provide a stimulus that has a low current drain on the power source of an implantable or small size held medical device with a self contained power source.

### Inventive Example 1A

Inventive Apparatus

A primary goal of the present invention is to provide a therapeutic and/or diagnostic electrical stimulus that uses less charge than presently available devices for both internal and external applications. The immediate gain is the ability to use a smaller size battery while providing the same or better efficiency than the larger size batteries. The apparatus of the present invention can provide just as clinically useful electrical stimulation and yet be the size of a pen. The smaller size is an advantage as it is critical in some clinical situations where there is limited space. This would be especially true when the apparatus of the present invention is incorporated into implanted medical devices. The present invention would employ digital/analog field programable gate array technology which further enhances the electrical efficiency over the prior art devices. Additionally miniaturized capacitors would be utilized. Device specific custom formed transformer cores may be employed. By using high frequency pulses to generate the stimuli the filtering properties of the capacitors and their size is less of a concern and miniaturization is feasible. The high frequency pulses used in the invention range from 1 kHz to 2000 kHz.

### Inventive Example 1B

In this Example which is an embodiment of the present invention one of the two electrodes is a very large dispersion electrode (either internally or externally placed on the body). The use of a very large dispersion electrode is to achieve a maximum conductance (or minimum resistance) at this electrode and reduce the voltage demand of the stimulator further enhancing the battery life reducing any possible physiologic effect at the placement site due to current density dispersion. The apparatus is enabled to use a high speed data link (for example, Blue Tooth technology) for control purposes. The apparatus may have a minimum of manual controls (for example an on/off switch, or even no manual switches) and respond to verbal commands from the operator. The microcircuitry for this apparatus may be manufactured using a surface mount board to minimize size demands.

### Inventive Example 1C

Method 1 of Inventive Example 1 is modified so that instead of 0.25 msec rest periods, a continuous waveform or carrier frequency is used.

### INVENTIVE EXAMPLE 2 (CARRIER FREQUENCY)

With a 1 cm in diameter electrode placed in front of each ear various frequencies of sinusoid waveform electrical stimulation was administered. The frequency of 40 Hz was able to evoke a non-cutaneous sensation of flashing or flickering lights in the periphery of the visual field. This non-cutaneous sensation was accompanied by a cutaneous sensation of electrical current or tingling at the site of the electrodes. Frequencies in the range of 2000 Hz which have on average a cutaneous Current Perception Threshold (CPT) greater than eleven (11) times the CPT of the 5 Hz stimulus at this site did not evoke any non-cutaneous sensations. When a 2000 Hz and a 2040 Hz stimulus were administered simultaneously the subjects reported the same non-cutaneous sensation as the 40 Hz stimulus but with no cutaneous evoked sensation.

### Inventive Example 2.1 (Bladder Placement, Etc.)

In this inventive example bladder dysfunction is addressed using an external electromagnetic power source, such as for, e.g., patient controlled treatment of spastic or paintful bladder conditions by attachment to the bladder.

Similar applications may be made, e.g., for bowel dysfunction or nerve dysfunction, for example to modulate nervous tissue function.

### Inventive Example 2.2 (Pancreatic Placement, Etc.)

An inventive device is placed to effect insulin release for the treatment of diabetes through the selective stimulation of pancreatic Islet cells to release insulin.

An inventive device also may be placed at various sphincters (e.g., Oddi, Pyloric, anal, etc.) to treat various types of related organ dysfunction of the gall bladder, stomach or bowel, respectively.

### Inventive Example 2.3 (Pelvic Pain and other Pain)

The invention may be used for treatment of pelvic and other types of pain by administration of the stimulus over nerve plexi or related spinal segments or CNS regions for diagnostic and/or therapeutic applications.

Although some conventional devices are available for the electrical treatment of pain, the present invention provides superior pain treatment, including smaller devices for neuro-selective stimulator which have advantageous safety and therapeutic efficacy as compared to conventional, non-neuroselective stimulator devices.

### INVENTIVE EXAMPLE 3

Referring to figures depicting schematic circuit diagrams in this specification, the following letters and designations are used as prefixes for certain circuit items identification numbers: Q for transistor, U for integrated circuit, R for resister.

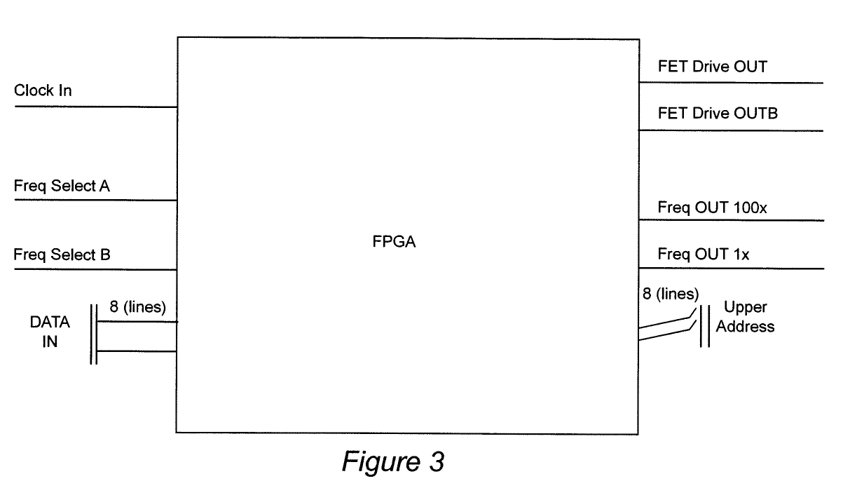

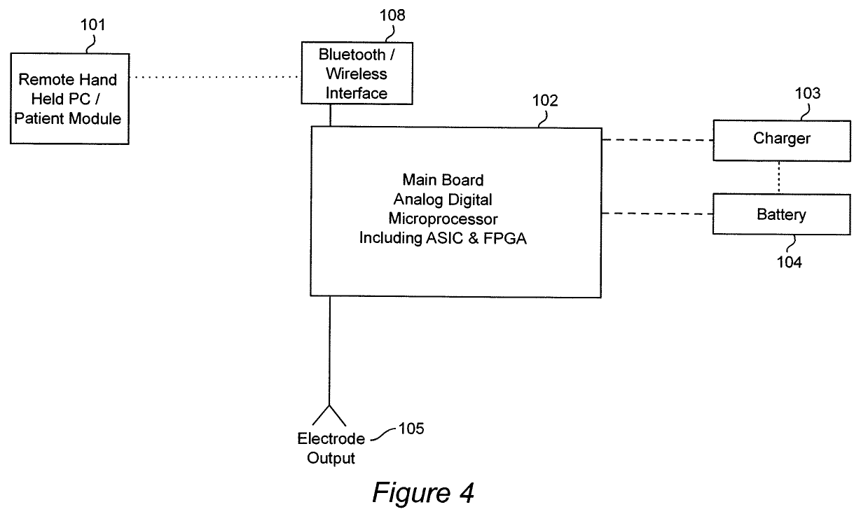

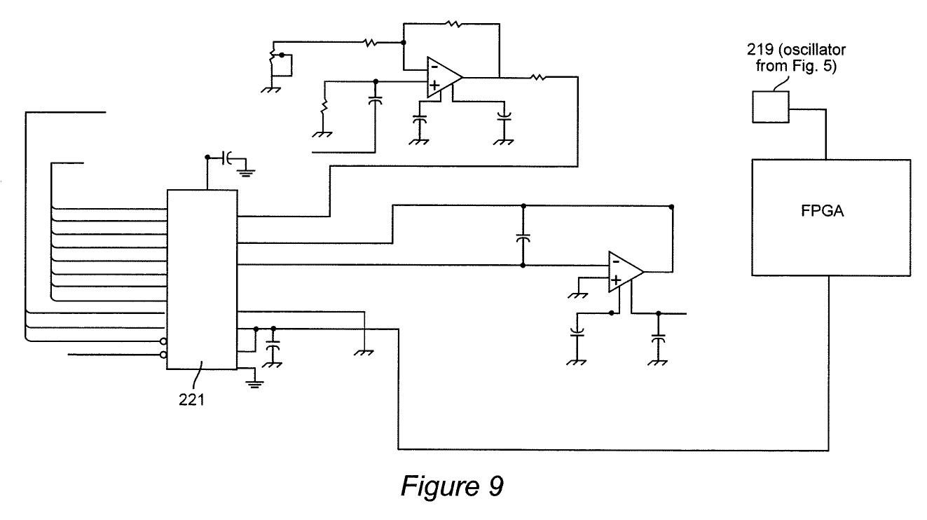

Referring to FIG. 4, the apparatus in this inventive example consists of the mainboard 102, containing both analog and digital circuitry, a microprocessor and an ASIC or FPGA chip. In FIGS. 3, 9 where FPGA is mentioned, an ASIC chip may be used instead. Referring again to FIG. 4, a remote handheld or laptop or similar personal computer includes software permitting a technician to control the device 9 and serve as a subject monitor or subject response module.

This Inventive Example does not require such a large battery as is required in Comparative Example 1A. For example, in this inventive example, the device may be powered using a lithium battery such as Sanyo lithium cell type 2CR5 which has weight 40 g, and dimensions 34 mm by 17 mm by 45 mm. In FIG. 4, battery 104 is shown but in other embodiments, an inventive device may be powered by other than a battery. An inductive device may or may not be battery operated but is designed so as not to be able to operate with line power so as to reduce the possible risk of electrical shock hazard.

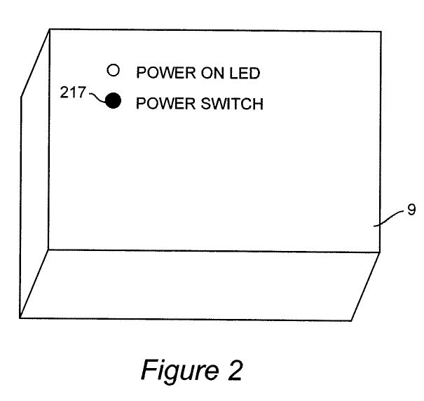

An example of a power source for the device 9 (FIG. 1D, FIG. 2), is, e.g., an internal battery as battery 104 (FIG. 4) for example a Sanyo Lithium Cell Type 2CR5 six (6) Volt battery measuring 34 mm (L)×17 mm (W)×45 mm (H) weighing 40 g. This is considerable smaller in contrast to the battery presently used in the current prior art devices describes in U.S. Pat. No. 5,806,522 a Panasonic Lead Acid Battery LC-R067R2P a 151 mm (L)×34 mm (W)×100 mm (H) battery with an approximate mass of 1.26 kg.

When an internal battery is used, the internal battery may be charged in various ways, such as by using an external battery charger that is connected to line power. The charger 103 (FIG. 4) may be, e.g., is a commercially available stand alone unit (e.g., Tamara, Inc., Japan). There is also a charger section on the mainboard 102 (FIG. 4). The charger in FIG. 4 also refers to an inductor which may be used for battery charging or device power.

Alternately, an internal battery may be charged using an external induction coil via electromagnetic energy transmission, as is a common means of recharging electric tooth brushes and certain health care devices.

In other embodiments, inventive devices may be operated without any internal battery source using and electromagnetic or similar energy source with the appropriate energy transducer mechanisms (e.g. a circular conductor) built into the device.

A wireless energy source facilitates the implantation of an inventive device into the body when medically indicated.

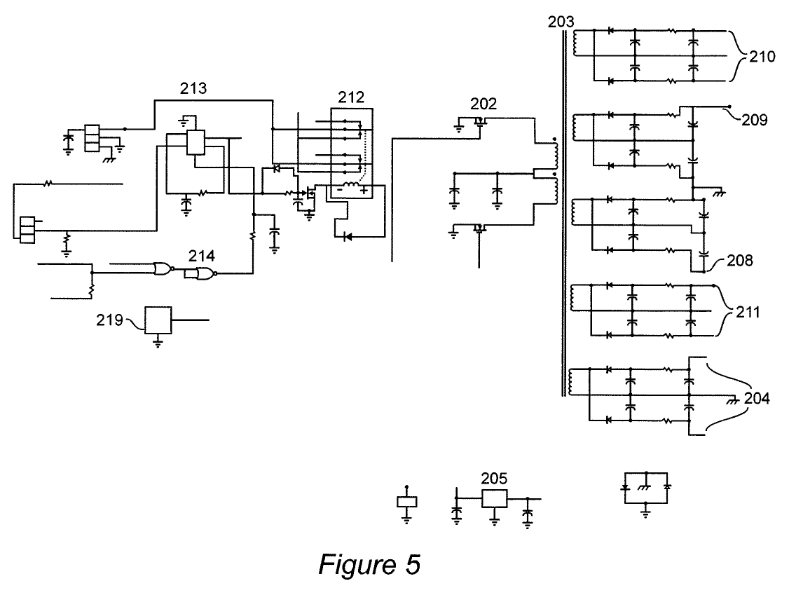

Referring to FIG. 5, the Power Supply Section (FIG. 5) receives 6 volt input from the battery 104 (FIG. 4). As a safety feature, the power supply (FIG. 5) is inherently limited through the use of small MOSFETS 202 (Ron>0.3 Ohms) and a small transformer 203 (<5 VA), thereby limiting the amount of power available at/ to the output. This provides an ultimate back-up safety feature. Under the failure of any other portions of this circuitry, there is not sufficient high voltage power available to harm the patient.

Power Supply Schematic (FIG. 5) is a component of the main board 102 (FIG. 4). The power supply section (FIG. 5) produces the necessary voltages from the 6 Volt (V) battery 104. It produces the plus and minus 14 V 204 for the analog circuitry, plus 5 V 205 for the digital circuitry, plus 5V and minus 5 V precision for the precision analog circuitry, plus 135 V 208 and minus 135 V 209 for the high voltage circuitry, and then two isolated plus and minus 15 V supplies each of which are referenced to the 135 V 208, 209 supplies, producing a plus 150 V 210 and a plus 120 V 210 centered around the plus 135 V 208 and a minus 150 V 211 and minus 150 V 211 centered around the minus 135 V 209.

The high speed design of the present invention permits the use of micro capacitors, significantly decreasing the size of the device. Referring to FIG. 5, the size of 10 large capacitors (2″ tall, 0.75″ diameter) which otherwise would be used in a conventional system near 210, 209, 209, 211 and 204. advantageously can be replaced in the present invention by micro capacitors or miniaturized capacitors, e.g., miniaturized capacitors as manufactured by Murata (www. murata. com). Oscillator 219 (FIG. 5) also is shown in FIG. 9.

The plus and minus 14 V 204 supplies power the low level analog circuitry. The plus 5 V reference supply is used to power the low level analog circuitry in the digital waveform synthesizer (FIG. 9). The power supply (FIG. 5) also has an on/off function. The actual power to the switching regulator (FIG. 5) and is passed through a relay 212. Relay 212 is controlled by an always powered CMOS flip/flop 213. CMOS flip/flop 213 detects activation or depression of the power on button 217 illustrated in FIG. 2.

In FIG. 2, the switch 217 is a membrane on /off switch which is located on the outside surface of an external model as shown in this Figure and labeled “Power Switch”. Alternatively, for internalized or implanted devices no external on/off switch or LED is required and electromagnetic communications can be wireless.

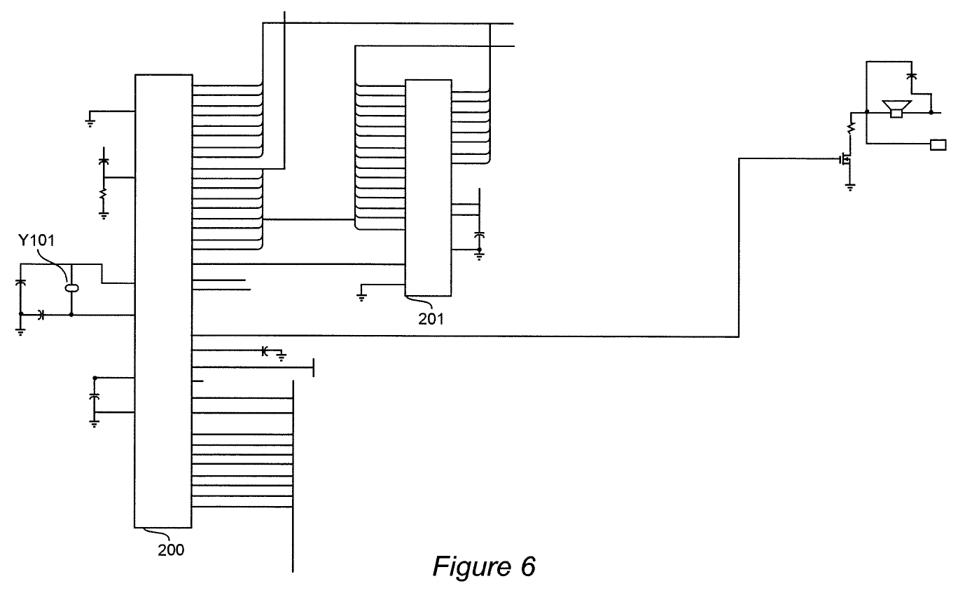

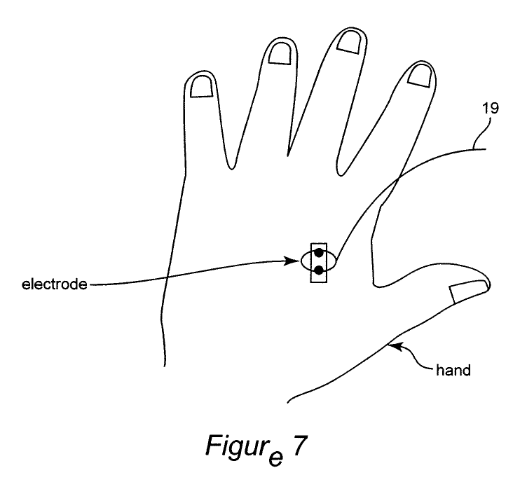

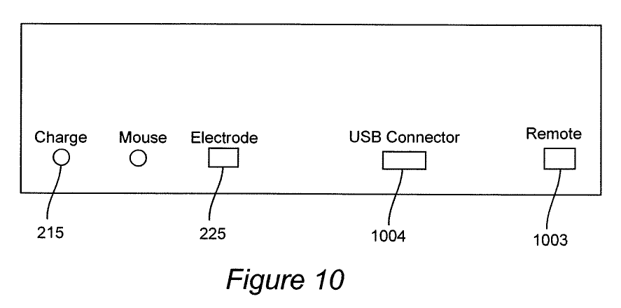

Referring to FIG. 5, the flip/flop 213 and associated logic circuitry 214 monitors the status of the charging jack 215 illustrated in FIG. 10. (FIG. 10 is optional for a sealed or internalized device.) If the extra set of contacts in the charging jack 215 are opened then the logic circuitry 214 resets the flip/flop 213 which forces the relay 212 to open and turns off the entire unit 9. This sequence may also be actuated by the micro-controller 200 illustrated in FIG. 6, thereby implementing the battery saving auto off function.

Referring to FIG. 5, alternatively, an inductive wireless power system may also be used.

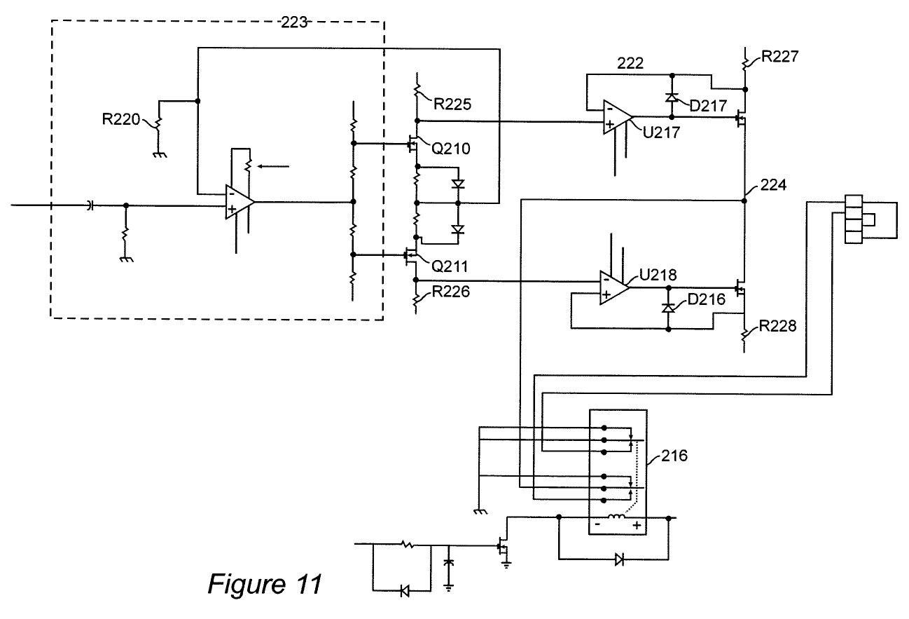

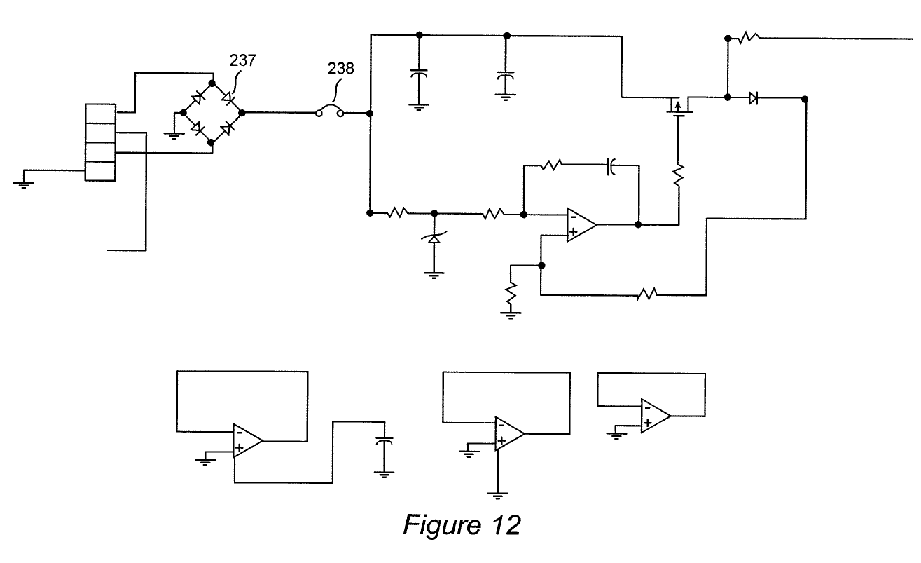

Referring to FIG. 11, an additional safety feature, is separate relay 216 from the power supply relay 212 illustrated in FIG. 5 controls the output signal. Relay 216 is switched on approximately one second after the power goes on. Relay 216 is switched off immediately when the on/off switch 217 is pressed to turn the unit (FIG. 4) off, while the actual power for the unit (FIG. 4) goes off approximately one second after the output relay 216. Therefore, the output relay 216 is never closed when the power is turned on or turned off, thereby preventing accidentally discharging the electrical stimulus to the patient 218 (illustrated in FIG. 1) or tissue while turning the device on or off. When using a line power charger this design ensures there are no start-up transients or turn-off transients. The output relay 216 also interrupts the output ground, so that in the unlikely but theoretically possible situation of the unit (FIG. 4) being hooked up to a failed and shorted charger 103 plugged into a wall outlet which was incorrectly wired, having the live and ground switched, and a patient connected who is touching a ground, there still will not be any hazard.

Referring to FIG. 5, the power supply is synchronized to the 2 megahertz quartz crystal 219 which is also used for the frequency generation system as illustrated in FIG. 9. The frequencies are generated by dividing the 2 megahertz crystal 219 until you generate frequencies at 100 times the desired the output frequency. The 500 Hz signal is generated to create the 5 Hz sinewave. Also generated is a 25 kHz signal to generate the 250 Hz sinewave and a 200 kHz is generated to create the 2 kHz sinewave. The Field Programable Gate Array (FPGA) or ASIC Chip 100X signal (FIG. 9) clocks a switched capacitor filter within the FPGA which is then divided by 100 and used to provide an analog input to its internal switched capacitor filter (within the FPGA). The switched capacitor filter extracts the fundamental frequency from the divided signal. This feature produces a very clean sinewave, which upon inspection appears to have greater than 1000 timing steps. Because the same path is followed by all three frequencies, there are no amplitude variations. Additionally, because each frequency is traceable back to the quartz crystal, the accuracy is that of the original crystal 219. The duration of stimulus and timing of the presentation is quartz crystal controlled by a different second crystal Y101 and the micro-controller 200 The analog signal generated from the frequency synthesis section illustrated in FIG. 9 is then amplified and applied to a multiplying Digital/Analog (D/A) convertor 221 (FIG. 9) under micro-controller 200 control. The multiplying D/A convertor 221 (FIG. 9) is a 14 bit unit. Therefore, it has 16,384 individual steps. The device in one embodiment uses the first 10,000 of these steps. In an alternative design, a 12 bit D/A convertor may be employed and the first 4,000 steps are used. The micro-controller 200 uses the extra steps for higher precision. The FPGA will generated the upper byte of the memory address. In one inventive example, 1,000 discrete codes are available to the user. After multiplying through the D/A convertor 221 (FIG. 9) to set a selected amplitude, the sinewave produced is fed to a transconductance amplifier (FIG. 11). The first section of the transconductance stage 223 creates two half copies of the signal, one is level shifted up to the high positive voltages and one is level shifted down to the high negative voltages. Current mirrors 222, whose gains are approximately 6.2 are used to produce output currents from the two half signals, which are then combined at the output 224. The output signal then goes through an output relay 216 to the output jack 225 (FIG. 10) Referring to FIG. 4, the communications interface circuitry 108 is concerned with interfacing with the PC (101). The processing in this Example is performed with an 8032 micro-controller 200 as illustrated in FIG. 6, using an offchip 201 memory of at least 16 kilobytes.

The battery voltage monitoring function is a micro-controller 200 (FIG. 6) controlled dual slope integration technique using one section of a quad comparator 231(FIG. 8) and an opamp 232 (FIG. 8) to measure the battery 104 voltage. Two sections of the quad comparator 233 (FIG. 8) provide clipping information.

Referring to FIG. 12, the main board incorporates a battery charger circuit if a battery charger 103 (FIG. 4) is present. A bridge rectifier 237 is provided on the charger input. This allows the use of a charger 103 (FIG. 4) with either center positive or center negative polarity. There is also a Polyfuse® current limiter device 238 (manufactured by Raychem of the USA), which takes the place of a fuse. The charger circuit (FIG. 12) takes the raw unregulated voltage being provided by the charger unit 103 and produces a precisely regulated 7 volt level for the battery 104 without the risk of overcharging, thereby significantly enhancing the life of the battery. The use of the bridge rectifier 237 and internal regulator (FIG. 12) also allows a wide variety of chargers to be used with the unit. This simplifies the production of units for operational capability using the various types of voltages found in many parts of the world.

Referring to FIG. 12 when a powering system comprising an induction coil is used, no battery is involved when an induction coil is used.

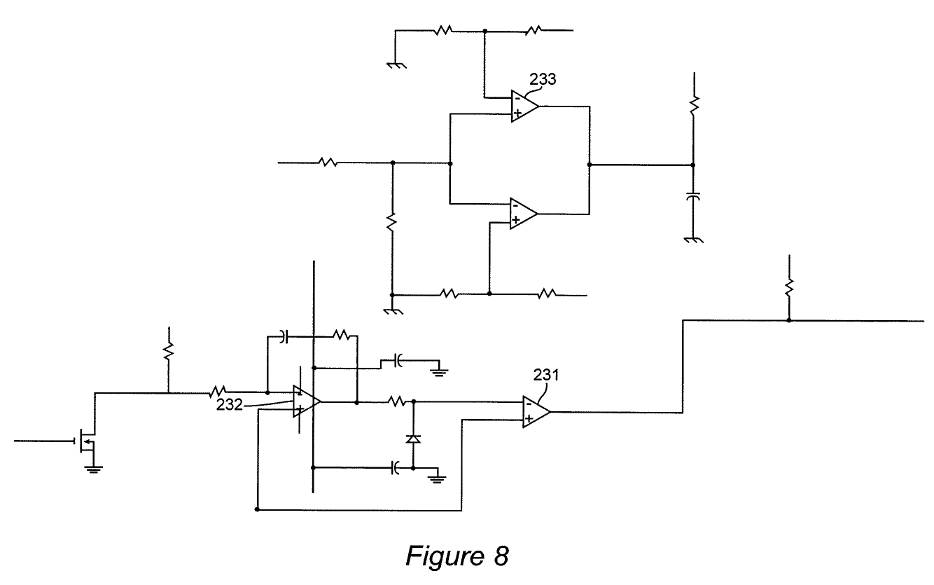

Referring to FIG. 6, microcontroller 200 includes a built-in controlled electrode test feature which can be executed before use of the unit 9, as shown in FIG. 2, to guarantee the integrity of the electrode cables 19 (FIG. 7) and check for shorts and opens. The microcontroller 200, in order to prolong battery life, automatically turns off the unit 9 after an operator set or default (e.g., 20 minute) duration of operational commands.

Referring to the figures discussed above, including FIG. 4, it will be appreciated that appropriate connections are established, depending on the particular system parts used, and that connections are not limited to what is specifically drawn. For example, referring to the electrode output 105 in FIG. 4, there may be used one connection for the electrode cable or, for example, four additional connectors for a charger 103, remote box connector 1003, mouse and USB connector 1004. These last four connectors are optional. The device advantageously may be Blue Tooth or WAN or IR or other wireless technology enabled.

The device of this Inventive Example, because of the high-frequency of the FPGA chip or ASIC chip, does not require as large capacitor(s) as in the device of the Comparative Examples. Therefore, the device in this inventive Example advantageously may use capacitors which are miniaturized compared to capacitors in any Comparative Example.

By using high frequency wave forms to generate the stimuli, the filtering properties of the capacitors and their size is less of a concern and miniaturization is feasible. The high frequency wave forms are in a range from 1 kHz to 50,000 kHz. The overall size (surface area) of capacitors on the circuit board in the device of this Inventive Example is reduced by 60% to 80% of the area occupied by capacitors in the Comparative Examples. Therefore, the overall size of the circuit board in inventive Examples 2 is greatly reduced compared to the Comparative Examples, as the associated surface mounted wiring to all the surface mount components is reduced as this wiring too is replaced by the FPGA or ASIC chip.

Digital Frequency, Waveform, and Duration Accuracy Improvement

A synthesized waveform is used. The synthesized waveform's accuracy is traceable back to the quartz crystal 219 inside the device 9. The frequency is virtually perfect for biomedical applications, i.e. it is in the order of several parts per million. The waveform is synthesized with a switched capacitor filter, so waveform purity is no longer subject to adjustments, calibrations or drifts as with conventional designs. The duration of presentation is controlled by a separate quartz crystal Y101 in a micro-controller 200 controlled sequence with similar accuracy, i.e. it is in the order of several parts per million.

Reduced Manufacturing Costs and Enhanced Reliability

There are several areas where manufacturing costs of an inventive apparatus have been reduced in comparison with conventional devices. A primary area is through the use of the FPGA or ASIC (FIG. 4). The previous technology was more labor intensive and expensive to effect.

Importantly and advantageously, the inventive medical device of this Iventive Example eliminates components (1) through (5) of Comparative Example 1A which otherwise occupy substantial circuit board space. An FPGA-based or ASIC-based device according to this Example permits miniaturization of Comparative Example 1A's signal generation circuitry by more than 500%. In avoiding components (1)-(5) of Comparative Example 1A, approximately 5.74 cm² of circuit board space are recovered; in using an FPGA microchip only 1 cm² of circuit board space is needed resulting in a net gain of 4.74 cm² of circuit board space by using the invention.

Additionally, the voltage demands of the FPGA or ASIC microchip compared to the conventional technology (Comparative Example 1) for generating the stimulus is approximately 50% more efficient in its voltage consumption. This feature facilitates device design and has the advantages of a small battery and other component size requirement and longer battery life over Comparative Example 1.

Another advantage of using the FPGA to generate the sinusoid stimulus waveform is that the waveform has less harmonic distortion (from digital noise) than the conventional technology (Comparative Example 1). The conventional technology is limited to generating a sinusoid waveform a maximum digital rate of 100 steps to generate 180 degrees of the waveform. The FPGA in the invention permits using rates of waveform generation over 1 thousand times faster (e.g. 100,000 steps in synthesizing the waveform).

### Example 3A (Operation of the Inventive Device of Inventive Example 3)

A device of Inventive Example 3 is connected to a patient (subject). Two sources of contact with the patient are needed for electrical testing.

The apparatus of inventive Example 3, being computer controlled, is capable of functioning in various output modes determined by the operator of the device through pressing switches on the control panel of the PC 101 (FIG. 4) for test or related device mode selection. Examples of these various modes of operation are as follows.

Referring to FIG. 1D, operation of an inventive system may be appreciated, such as an initial start-up mode of operation. A remote module or PC 101 is in use by an operator 107 and subject 218. The dimensions of the PC 101 are approximately 9 cm×6 cm×1.5 cm. The dimensions of the inventive device 9 (FIG. 1D) in this inventive Example is a hand-held size of about 5 cm×5 cm×2 cm. The dimensions may vary depending upon the configuration, which is application specific. Alternatively, a separate additional PC may be used.

After the technician 107 (FIG. 1D) presses the power button 217 (FIG. 2) and turns on the device 9 (FIGS. 1D, 2), the remote hand held personal computer (PC) 101 display displays information, such as identifying the manufacturer of the device and any related information regarding identification of the device and typical display screens and controls of modes of operation associated with neuroselective sensory nerve conduction devices. The technician 107 may select the mode of operation from the PC (101).

Subject control via PC 101. After receiving instruction in conducting the evaluation from the PC 101 or the tester, the subject 218 (FIG. 1D) selects the test with its accompanying intensity alignment choice from the PC 101 display. The display typically is touch sensitive and the PC 101 may have a built-in video CAM, microphone and speakers. This subject controlled alignment procedure is conducted by the subject 218 using the PC 101. The subject 218 is instructed or receives a visual and/or auditory cue to press and hold the switch labeled on the PC display screen 101 until the electrical stimulus is perceived from their body site in contact with the electrodes and follow the instructions associated with the instructions and virtual buttons on the PC 101 display. Alternatively the speakers in the PC device 101 may issue audio instructions or a microphone built in or attached to PC device 101 may be employed to monitor the patient's verbal or auditory responses. Additionally, other types of physiological measures may be monitored including brain responses using functional magnetic resonance imaging or Positron Emission Tomography. Additionally, alternatively, physiological measures may be ascertained using the present invention, such as in conjunction with physiological monitoring to measure physiological responses to the electrical stimulation. This may be incorporated, for example, intraoperatively in surgery in assessing sensory function in patients suffering from intractable pain and other neuropathological conditions such as syringomyelia. The information obtained by the clinician in monitoring peripheral nerve cells responses to this type of electrical stimulus that is standardized is valuable for prognostic purposes and in guiding the surgeon as to which nerve tissue is pathological for biopsy purposes, ablation purposes and for pharmaceutical treatment purposes, as well as electrical stimulation for therapeutic application purposes.

### INVENTIVE EXAMPLE 4

The inventive machine of this example is about 0.2-6 pounds, and dimensioned about 6 inches (L), 6 inches (W), 1 inch (H), or a 3 inches by 3 inches by 2 inches cube or oval shape. The machine of this example has 1 switch, and may be mechanically or electrically activated. The following connectors are optional: 1 USB; 1 telephone (TELCO 6-4); 1 remote box (TELCO 8-8); 1 charger (CD connectors 2.1-2.5 mm); mouse (ADB) connector. An internal battery is optional.

Power-on LED is optional, depending on the intended application. For example, if the machine is to be implanted, a touch turn-on button would not be wanted. Preferably, the on/off switch is integrated. For example, the switch 217 may be a membrane on/off switch located on the outside surface of an external model as shown in FIG. 2 and labeled “Power Switch.”

This machine is designed to work with a laptop computer or hand held PC via a Blue Tooth connection or other appropriate connection, including, e.g., 802.11-G (WAN) or other wide area network. An Hewlett Packard touch-sensitive screen may be used, to provide virtual buttons for a patient (subject) to touch. The PC may be the same as the remote box, or may be separate from the remote box.

This machine may replace optical isolation with magnetic isolation for connectors. Using magnetic isolation is preferred, to use less board space.

This machine may use an induction coil instead of a battery charger.

This machine may use a custom-shaped wire wound toroid rather than a conventional transformer or a wound wire induction coil.

While the invention has been described in terms of its preferred embodiments, those skilled in the art will recognize that the invention can be practiced with modification within the spirit and scope of the appended claims.

What I claim as my invention is:

1. A medical device comprising:
a generator system comprising a field programmable gate array (FPGA) chip or a application-specific integrated circuit (ASIC) chip; wherein the generator system generates at least one stimulus which is a continuous symmetric wave form; and
at least one electrode or electromagnet system via which the at least one generated stimulus may be administered to a patient or an electrosensitive tissue.

2. The medical device of claim 1, wherein the FPGA chip or ASIC chip is a high-frequency chip in a range of about 1 kHz to 50,000 kHz.

3. The medical device of claim 1, wherein the symmetric wave-form is a sine-wave.

4. The medical device of claim 1, wherein the device is hand-held or smaller and/or weighs substantially less than 33 pounds and/or has dimensions no bigger than 15 cm by 15 cm by 10 cm.

5. The medical device of claim 1, wherein during operation of the device an amount of current required is less than 20 mAmp.

6. The medical device of claim 1, consisting essentially of:
the high-frequency FPGA chip or the high-frequency ASIC chip, and only such additional components as are necessary to operate a constant current test when the device is electrically connected to a patient or a tissue.

7. The medical device of claim 1, including a power source.

8. The medical device of claim 7, wherein the power source is a battery.

9. The medical device of claim 1, powered by an inductance coil.

10. The medical device of claim 1, powered by an external power source not included in the device.

11. The medical device of claim 1, wherein the at least one stimulus generated is of a form that can be applied to electrosensitive tissue.

12. The medical device of claim 11, wherein the electrosensitive tissue is within a human or an animal.

13. The medical device of claim 1, biocompatilized and implantable into a human patient.

14. The medical device of claim 1, wherein the generated at least one stimulus when applied to a patient elicits no cutaneous sensation and only non-cutaneous sensation.

15. The medical device of claim 1, wherein at least one wave form is generated for a time in range of about 1 second to several minutes.

16. The medical device of claim 1, wherein the generated stimulus is neuroselective or tissue selective.

17. A medical device comprising:
a generator system that generates a particular harmonic frequency by maximizing at least two or more different frequencies which differ from the particular harmonic frequency;
at least one electrode or electromagnet system via which the particular harmonic frequency may be administered to a patient or an electrosensitive tissue.

18. The medical device of claim 17, wherein the particular harmonic frequency is neuroselective or tissue selective.

19. The medical device of claim 18, wherein the particular harmonic frequency is selective among a subpopulation of nerve fibers selected from the group consisting of A, B and C nerve fibers.

20. The medical device of claim 17, wherein the particular harmonic frequency is capable of selective stimulating different tissue types.

21. A miniaturized medical device for generating a stimulus receivable by electrosensitive tissue, comprising:
a stimulus-generating system that generates a stimulus; and
an electrode or electromagnet system through which the stimulus can be delivered to electrosensitive tissue,
wherein the device is a size that is hand-held or smaller.

22. The miniaturized device of claim 21, wherein the stimulus is a continuous symmetric wave-form.

23. The miniaturized medical device of claim 21, wherein the device has a weight substantially less than 33 pounds.

24. The miniaturized medical device of claim 21, wherein the apparatus is implantable into a human or animal.

25. A method of generating medically-useable electrical stimulation, comprising:
within a device of a size that is hand-held or smaller, generating at least one electrical stimulus having a continuous symmetric waveform and having a high frequency in a range of about 1 kHz to 50,000 kHz;
providing the at least one electrical stimulus to an electrode or electromagnet system wherein the electrode or electromagnet system is contactable with an electrosensitive tissue or a patient.

26. The method of claim 25, wherein the generating step comprises operating a high-frequency FPGA chip or a high-frequency ASIC chip.

27. A method of electrically stimulating electrosensitive tissue, comprising:
within a device of a size that is hand-held or smaller, generating at least one electrical stimulus having a continuous symmetric wave form;
applying the at least one electrical stimulus to an electrosensitive tissue.

28. The method of claim 27, including generating a wave-form in a range of up to 50,000 kHz.

29. The method of claim 27, including generating a wave-form in a range of about 1 kHz to 50,000 kHz.

30. The electrical stimulation method of claim 27, wherein the applying step comprises contacting a stimulating electrode with a patient who may be human or animal, or an electrosensitive tissue.

31. The method of claim 27, wherein the step of applying the at least one electrical stimulus is performed cutaneously.

32. The method of claim 27, wherein the step of applying the at least one electrical stimulus is performed non-cutaneously.

33. The method of claim 27, wherein the step of applying the at least one electrical stimulus is performed for a time in a range of about 0.1 second to several minutes.

34. The method of claim 27, wherein the step of applying the at least one electrical stimulus results in nerve or tissue stimulation.

35. The device of claim 1, controllable via a virtual switch on a personal computer, the virtual switch and the personal computer being separate from the device.

36. The device of claim 1, including a wireless interface.

37. The device of claim 36, wherein the wireless interface is selected from the group consisting of a Bluetooth wireless interface, a WAN wireless interface, an 802.11-G (WAN) wireless interface, and infrared.

38. The method of claim 27, including a step of minimizing charge density, for safety.

39. The device of claim 1, including a wire wrapped toroid or a custom-wrapped electro-conductive coil.

40. The device of claim 1, which cooperates with an external wire wrapped toroid not contained in the device itself or a custom-wrapped electro-conductive coil not contained in the device itself.

41. The method of claim 27, wherein the step of application of the stimulus to the tissue is for one selected from the group consisting of controlling internal muscular action, bladder therapy, stomach therapy, evoking selective insulin release for treatment of diabetes, other pancreas therapy, and pain management.

42. The device of claim 1, having no battery within the device.

43. The device of claim 1, comprising at least one capacitor and wherein any capacitor in the device is a micro-chip capacitor.

44. The device of claim 17, including at least one other electrode which is a return electrode, a skin dispersion electrode or a combination thereof.
